(19) United States
(12) Patent Application Publication
(10) Pub. No.: US 2023/0122353 A1
(43) Pub. Date: Apr. 20, 2023

(54) COMPUTER-IMPLEMENTED SYSTEMS AND METHODS FOR COMPUTING PROVIDER ATTRIBUTION

(71) Applicant: Dignity Health, San Francisco, CA (US)

(72) Inventors: Ingrid Wurpts, San Francisco, CA (US); Joseph Colorafi, San Francisco, CA (US); Angelica Chanco, San Francisco, CA (US); Sunilkumar Kakade, San Francisco, CA (US); Mark Page, San Francisco, CA (US); Saurabh Bhutyani, San Francisco, CA (US); Monica Spoerer, San Francisco, CA (US)

(21) Appl. No.: 17/905,405

(22) PCT Filed: Mar. 19, 2021
(86) PCT No.: PCT/US2021/023285

§ 371 Date: Aug. 31, 2022

Related U.S. Application Data
(60) Provisional application No. 62/991,711, filed on Mar. 19, 2020.

(51) Int. Cl. G16H 40/20; G06N 20/00

(52) U.S. Cl. CPC G16H 40/20; G06N 20/00; G16H 10/60

(57) ABSTRACT

Various embodiments of a computer-implemented system for generating an algorithm configured to compute an attribution output to enhance provider attribution are disclosed.

### CROSS REFERENCE TO RELATED APPLICATIONS

The present document a PCT patent application that claims benefit to U.S. provisional application Ser. No. 62/991,711 filed on Mar. 19, 2020, which is herein incorporated by reference in its entirety.

### FIELD

The present disclosure generally relates to systems and methods associated with data analysis and computations in the field of healthcare; and in particular, to a computer-implemented system and methods thereof that leverages electronic health record documentation to assign a ranking to physicians based on predetermined parameters for provider attribution.

### BACKGROUND

Hospital administrators and executives often need to attribute a patient's care to one primary physician. The current method of attribution (attending at discharge) often means that a physician who was not substantively involved with a patient's care my receive attribution. In other words, patients are usually attributed to the provider who was listed in the electronic health record (EHR) as attending at discharge, despite other physicians potentially playing an important role in the patient's treatment and care.

It is with these observations in mind, among others, that various aspects of the present disclosure were conceived and developed.

### SUMMARY

Aspects of the present disclosure may be embodied as or take the form of a computer-implemented/executable method performed by at least one processor or processing element. Steps of the method include accessing, by a processor, a training dataset including electronic health record (EHR) data defining predefined decisions for physician attribution; and training, by machine learning conducted by the processor, a machine learning algorithm to learn and model the predefined decisions for physician attribution in view of the training dataset such that the processor executing the machine learning algorithm is configured to predict physician attribution from subsequent EHR data, by: inputting a plurality of variables associated with the predefined decisions of the physician attribution to a machine learning model, learning a set of predictor parameters for the machine learning algorithm that improve physician attribution prediction, and weighting and summing one or more fields of the machine learning algorithm in view of an accuracy threshold. The machine learning algorithm as trained, when fed with the subsequent EHR data defining a patient encounter, improves attribution data analysis by computing an attribution output based on the set of predictor parameters as learned.

The present disclosure further includes an embodiment of a system for computing physician attribution, comprising a first computing device, the first computing device having access to electronic healthcare records (EHR) data; and a second computing device in operable communication with the first computing device. The second computing device includes a processor configured to: access the EHR data from the first computing device, generate a training dataset from the EHR data including predefined decisions of physician attribution, input a plurality of variables associated with the predefined decisions of the physician attribution to a machine learning model, and generate and train a machine learning algorithm based on the machine learning model such that the machine learning algorithm is configured to learn the predefined decisions of physician attribution and is configured to predict physician attribution from subsequent EHR data. The system can further include an interface for visualizing aspects of the machine learning predictions and computations; e.g., attribution scores from application of subsequent EHR data to the machine learning model can be presented via the interface to illustrate predicted attribution based on an encounter associated with the subsequent EHR data.

The present disclosure further includes an embodiment of a tangible, non-transitory, computer-readable media having instructions encoded thereon, the instructions, when executed by a processor, are operable to: access EHR data, generate a training dataset from the EHR data including predefined decisions of physician attribution, input a plurality of variables associated with the predefined decisions of the physician attribution to a machine learning model, and train a machine learning algorithm based on the machine learning model such that the machine learning algorithm is configured to learn the predefined decisions of physician attribution and is configured to predict physician attribution from subsequent EHR data. Additional embodiments are contemplated. Aspects of the functionality described herein can be performed in a cloud or cloud-based infrastructure such that multiple processing elements contribute to the machine learning steps and application described herein.

### BRIEF DESCRIPTION OF THE DRAWINGS

FIG. 1 is a simplified block diagram of a computer-implemented system for generating and implementing an algorithm for physician attribution ranking.

FIG. 2 is an exemplary process flow for training, optimizing, and implementing the algorithm/model using machine learning to rate or rank a physician's attribution to a patient interaction or encounter.

FIG. 3A is a simplified block diagram illustrating machine learning and application of the SMART algorithm described herein.

FIG. 3B is an image illustrating functionality associated with generating model-selected predictors for physician attribution based on predetermined variables entered in or fed to a computer-implemented machine learning/statistical algorithm/model.

FIG. 4A is a graph illustrating that computing and extending attribution to a physician is generally preferable to merely extending attribution to an attending at discharge for both physician communication and length of stay.

FIG. 4B is a graph illustrating comparison between extending attribution to an attending versus extending attribution to a particular physician based on implementation of the SMART provider algorithm described herein.

FIG. 5A is an exemplary screenshot of a graphical interface for implementing aspects of the provider attribution computations described herein.

FIG. 5B is an exemplary screenshot of a graphical interface for implementing aspects of the provider attribution computations described herein.

FIG. 6 is an example simplified schematic diagram of a computing device that may implement various methodologies described herein.

Corresponding reference characters indicate corresponding elements among the view of the drawings. The headings used in the figures do not limit the scope of the claims.

### DETAILED DESCRIPTION

Aspects of the present disclosure relate to a computer-implemented system and methods thereof for leveraging data associated with healthcare including electronic health records to compute, using machine learning and applying one or more predetermined predictor parameters, an attribution output including a ranking of physicians and other outputs and metrics informative as to provider attribution. For reference and context, hospital systems seeking to improve overall patient outcomes need to utilize patient-level data yet face various technical challenges. For example, it is not always clear who is directly responsible for a patient's care and this information is difficult to interpret and process. As an example, in reducing Length of Stay (LOS), it is important to understand which physician(s) are making the decisions that affect a patient's LOS. Most patients will see a variety of providers during their stay in the hospital. Often, the discharge attending physician is given attribution for a patient's outcomes, although there is no guarantee that he or she was most involved in that patient's care. Moreover, current data points associated with LOS and interpretative functions are either nonexistent, difficult to access or process, or technically insufficient.

The present computer-implemented systems and methods provide a technical solution that utilizes data-mining methods that contribute to the development and implementation of an algorithm, designated “SMART (Provider Attribution (SPA)) algorithm.” The SMART algorithm described herein, as formed via machine learning and executed, computes an output that generally provides attribution to the physician responsible for communication and LOS for each discharged patient, based on inputs such as clinical documentation. Whereas the attending at discharge physician may have only had minimal interaction with the patient, the algorithm described herein leverages data structures from notes and orders that the physicians create, so any physician attributed as the primary physician has the majority of documented interaction with the patient. Hospital leaders can use the attribution information to better determine opportunities for improvement.

During post-implementation analysis and testing, the algorithm showed improved attribution versus using the discharge attending provider. Specifically, when assessing the performance of the algorithm, the positive predictive value (PPV=probability that physicians identified by the algorithm were identified as primary by the abstractors) was 89% for physician communication, 77% for length of stay. The PPV for attending at discharge was 65% for physician communication, 56% for length of stay. Accordingly, the algorithm improves attribution data analysis by computing an attribution output based on the set of predictor parameters as learned during machine learning.

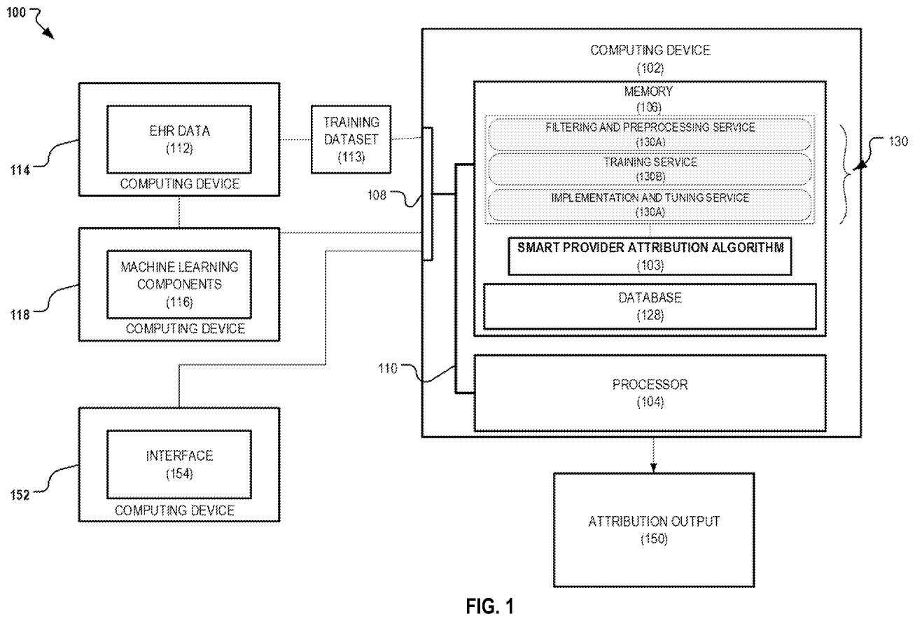

Referring to FIG. 1, a computer-implemented system is illustrated which may be implemented to assess provider attribution (hereinafter “system 100”). The system 100 includes a computing device 102 configured to develop, modify, and implement a Smart Provider Attribution algorithm, designated algorithm 103, and other functionality described herein. In general, the computing device 102 includes a processor 104, a memory 106 of the computing device 102 (or separately implemented), a network interface (or multiple network interfaces) 108, and a bus 110 (or wireless medium) for interconnecting the aforementioned components. The network interface 108 includes the mechanical, electrical, and signaling circuitry for communicating data over links (e.g., wires or wireless links) within a network (e.g., the Internet). The network interface 108 may be configured to transmit and/or receive data using a variety of different communication protocols, as will be understood by those skilled in the art.

As indicated, via the network interface 108 or otherwise, the computing device 102 is adapted to access electronic health record (EHR) data, designated EHR data 112, historical or new, which may be stored/aggregated within a memory of a computing device 114 (or locally stored within the memory 106). The EHR data 112 includes or defines clinical documentation or other such information (including physician notes, orders, relationships, and other data points) associated with patient encounters and historical records of the EHR data 112. The EHR data 112 may be of any form or format, and may include any number of type of data structures. The EHR data 112 may be filtered and/or preprocessed to normalize the EHR data 112 for machine learning and/or further applications.

The EHR data 112 may be utilized to generate a training dataset 113 as further described herein. The training dataset 113, based on the EHR data 112, is leveraged by the computing device 102 to train a machine-learning based algorithm 103 and/or generate functions, or rules suitable for assessing physician attribution and generating physician attribution rankings or scores, as further described herein. In addition, the computing device 102 may access or otherwise leverage any number of machine learning components running internally and/or machine learning components 116 hosted or otherwise stored temporarily on a computing device 118, such as a device of a machine-learning cloud system that provides access to machine learning models as part of a provided service (e.g. Amazon), or any other machine learning utilities such as training sets and other information. The system 100 is not limited to the components of FIG. 1 and may include other devices or sub-systems to supplement the computing device 102 with any other forms of information suitable for generating and optimizing the algorithm 103.

The EHR data 112 and the training dataset 113 is aggregated or accessed by the computing device 102 and may be organized within a database 128 stored within the memory 106. Once this data is accessed and/or stored, the processor 104 is operable to execute a plurality of services 130 to process the EHR data 112 and/or the training dataset 113 using one or more machine learning models/algorithms provided by the computing device 118 or otherwise accessed. The services 130 of the system 100 may include, without limitation, a preprocessing service 130A which may include machine-learning functionality for estimating parameters or predictors or extracting features from the EHR data 112 and/or the training dataset 113 based on one or more variables of the EHR data 112, a training service 130B for training the algorithm based on the parameters, and an algorithm implementation and tuning service 130C for implementing, evaluating, and tuning the algorithm 103 in any form, as further described herein. The plurality of services 130 may include any number of components or modules executed by the processor 104 or otherwise implemented. Accordingly, in some embodiments, one or more of the plurality of services 130 may be implemented as code and/or machine-executable instructions executable by the processor 104 that may represent one or more of a procedure, a function, a subprogram, a program, a routine, a subroutine, a module, an object, a software package, a class, or any combination of instructions, data structures, or program statements, and the like. In other words, one or more of the plurality of services 130 described herein may be implemented by hardware, software, firmware, middleware, microcode, hardware description languages, or any combination thereof. When implemented in software, firmware, middleware or microcode, the program code or code segments to perform the necessary tasks (e.g., a computer-program product) may be stored in a computer-readable or machine-readable medium (e.g., the memory 106), and the processor 104 performs the tasks defined by the code.

As further shown in FIG. 1, embodiments of the system 100 include a computing device 152, such as a client or administrator device in the form of a mobile device, a general computing device or laptop, a server, or the like. The computing device 152 executes an interface 154 based on data provided by the computing device 102. For example, data analysis computations and reporting can all be made accessible to physician accessing the interface 154 via the computing device 152. The interface 154 is non-limiting and may be offered as software-as-a-service SaaS accessible via a browser or application executed by the computing device 152.

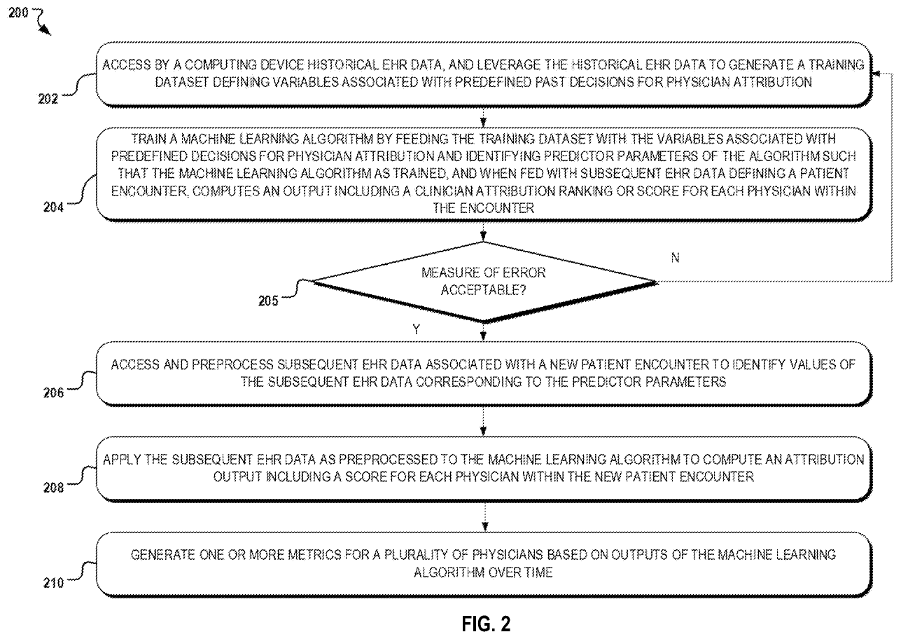

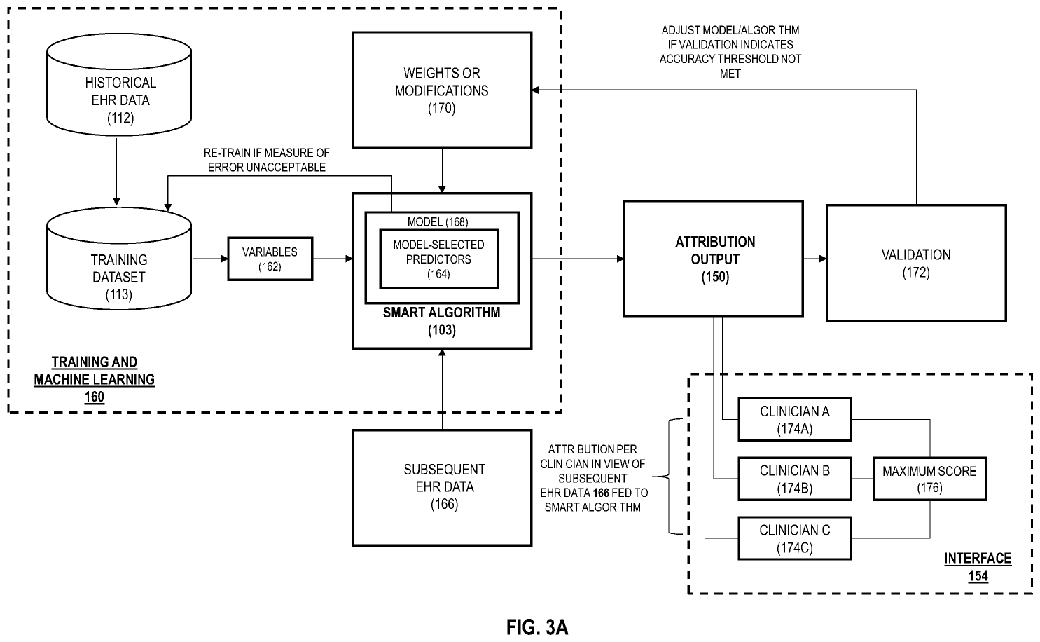

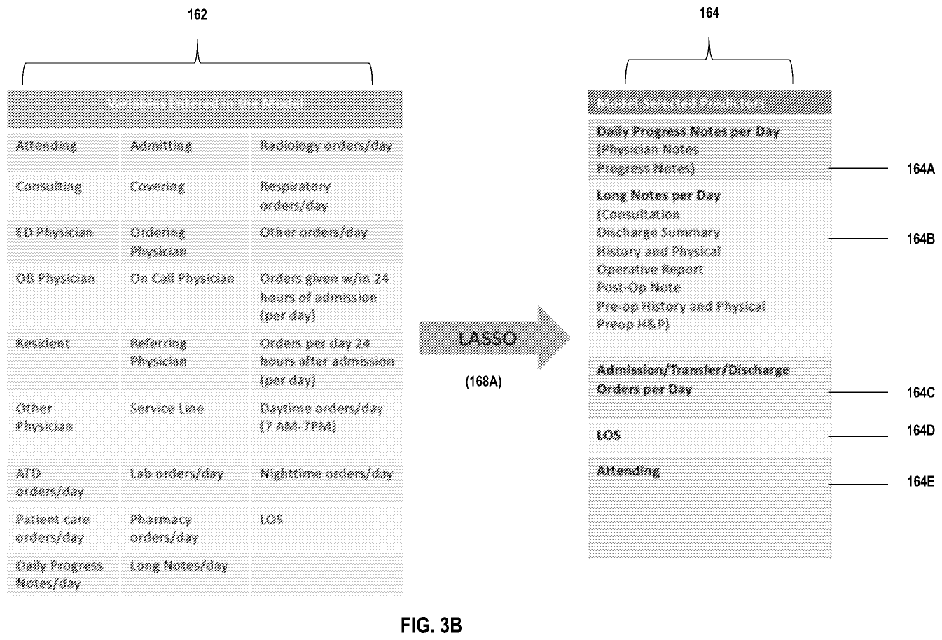

Turning now to a process flow diagram 200 referencing FIG. 2, with reference to FIG. 3A and FIG. 3B, further aspects regarding the implementation of the system 100 shall now be described. Referring to block 202 of FIG. 2, the computing device 102 is implemented to execute training and machine learning 160 to ultimate select and tune the predictors and characteristics of the algorithm 103. As described, the EHR data 112 comprises historical EHR records such as clinical documentation or other such information (including physician notes, orders, relationships, and other data points) associated with patient encounters and historical records.

As indicated in FIG. 3A, the training dataset 113 (accessed by the computing device 102) is at least partially built upon the EHR data 112. In some embodiments, the training dataset 113 is based on known, pre-accepted, or predefined decisions where one or more physicians was properly attributed for a patient encounter, in view of a set of variables 162, as extrapolated from or built upon from the EHR data 112. For example, FIG. 3B shows a variety of possible types of the variables 162 that can be entered into the algorithm 103 during training and machine learning 160; by non-limiting examples, variables 162 may include length of stay (LOS), orders per day, a referring physician, etc. Instances of the variables 162 may relate to a patient encounter record of the EHR data 112 where a physician was properly attributed for the encounter. For example, the EHR data 112 may include a record where a physician P was properly attributed for a patient encounter. The variables 162 and associated values corresponding to the patient encounter from the Physician P provide some intelligence, pattern, or training information that can be leveraged during training of the training and machine learning 160, so that the computing device 102 executing the algorithm 103 learns why the Physician P was attributed and predictor parameters (164) can be selected and adjusted accordingly during machine learning.

Referring to block 204, the computing device 102 may train a machine learning algorithm or model to ultimately derive a trained algorithm, i.e., the algorithm 103, using the training dataset 113, and additional machine learning training or validation (172) sessions may be iteratively conducted using the computing device 102 to optimize the algorithm 103. The algorithm 103, properly trained, includes a set of specific predictor parameters 164, fields, or variables (which may be previously unknown), weights, and mathematical functionality (based on known attribution decisions provided by the training dataset 113) that can be executed by the computing device 102 to estimate physician attribution for an encounter, and estimates physician attribution by computing an attribution output 150, which may define a score, ranking, or other metric for each physician associated with a new encounter based on subsequent EHR data (166B) associated with the new encounter. The algorithm 103 may define or be assigned a predetermined measure of error or accuracy threshold to determine when the algorithm 103 is deemed to be optimal for real-world use when fed with subsequent EHR data 166.

To train the algorithm 103 during the training and machine learning 160, in some embodiments, an initial machine learning model 168 or method (or plurality of models) is selected, such as a regression algorithm, and the computing device 102 applies the training dataset 113 to the model 168 to ultimately generate and/or select the set of predictor parameters 164 as the model 168 is fed with the plurality of variables 162 defined by the underlying EHR data 112. As such, the model 168 models relationships between the plurality of variables 162 and/or the set of predictor parameters 164, and is trained and tuned to ultimately derive the machine learning algorithm 103 by the computing device 102. During training, the model 168 may be iteratively refined by adjusting the model 168 in view of a predetermined measure of error associated with attribution output 150. In a specific example shown in FIG. 3B, a least-absolute-shrinkage-and-selection-operator (LASSO) model 168A is applied to the training dataset 113 to estimate the set of predictor parameters 164. In this embodiment, LASSO regression may result in the selection of the set of predictor parameters 164 via leave-one-encounter-out cross validation, and all two-way interactions may be considered. Training and tuning the model 168 during training and machine learning 160, the processor 104 of the computing device 102 ultimately forms the algorithm 103.

The model 168 described herein may include any form of regression model such as linear regression, logistic regression, polynomial regression, stepwise regression, ridge regression, LASSO regression, or ElasticNet regression. However, the model 168 is not limited to regression and my also include models from supervised learning, unsupervised learning, semi-supervised learning, or reinforcement learning such that the model 168 may be a classification model. In addition, the model 168 and/or the machine learning algorithm 103 may include any number of models, algorithms, or equations running sequentially or in parallel. In either case, the model 168 is generally applied by the processor 104 of the computing device 102 to learn relationships between the plurality of variables 162 and select features of the parameter-predictors 164 for the machine learning algorithm 103 so that the machine learning algorithm 103 produces the attribution output 150 predicting attribution of a physician.

As indicated in FIG. 3B, in view of machine learning and as the algorithm 103 is ultimately formed from the model 168, the set of predictor parameters 164 may be model-selected and include a variety of possible parameters learned during training. By non-limiting examples, the set of predictor parameters 164 includes progress notes per day 164A, long notes per day 164B, orders per day 164C, a length of stay (LOS) 164D, and an attending parameter 164E, and the like. The set of predictor parameters 164 and/or other fields of the algorithm 103 may be configured for any data type such as characters, Booleans, numbers, and the like.

In addition, during training, one or more of weights or modifications 170 may be applied to the predictor parameters 164 or fields of the algorithm 103 such that aspects of the algorithm 103 may be weighted and/or summed to improve the ability of the algorithm 103 to output the attribution output 150 in a manner that is consistent with the logic of the predetermined attribution decisions of the training data 113 (e.g., abstractor decisions used to form the training dataset 113). As indicated by the decision block 205, the algorithm 103 may be tuned, modified, or further trained as needed until the measure of error of the physician ranking output 150 is deemed to be acceptable.

Referring to block 206 and block 208 of FIG. 2, in general, the machine learning algorithm 103 as trained, when fed with subsequent EHR data 166 defining a new patient encounter, computes an attribution output 150 which may include a physician ranking output or score and/or one or more metrics for each physician within the new encounter. The attribution output 150 improves attribution data analysis by because the attribution output 150 as computed is based on the set of predictor parameters 164 derived through machine learning and extrapolates beyond the base EHR data 166 of the encounter.

One example of a physician ranking output 150 is provided below in the form of a score, designated Example Score:

Score=0,306*(Daily Progress Notes)+0.253*(Attending×Daily Progress Notes)+0.0633*(LOS×ATD Orders)+0.0747*(LOS×Long Notes)

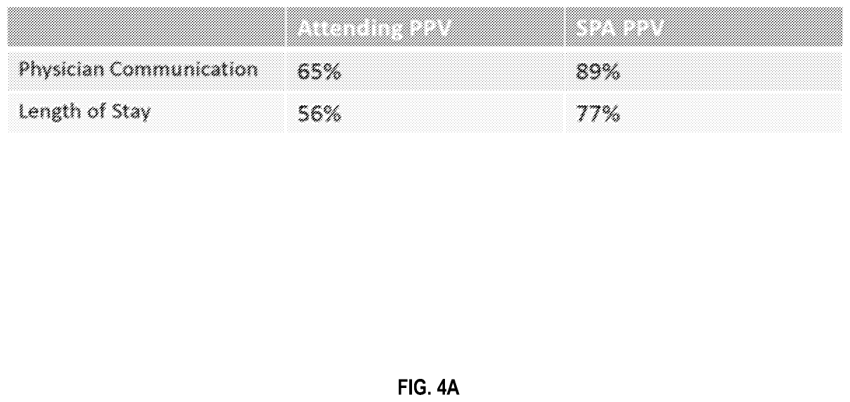

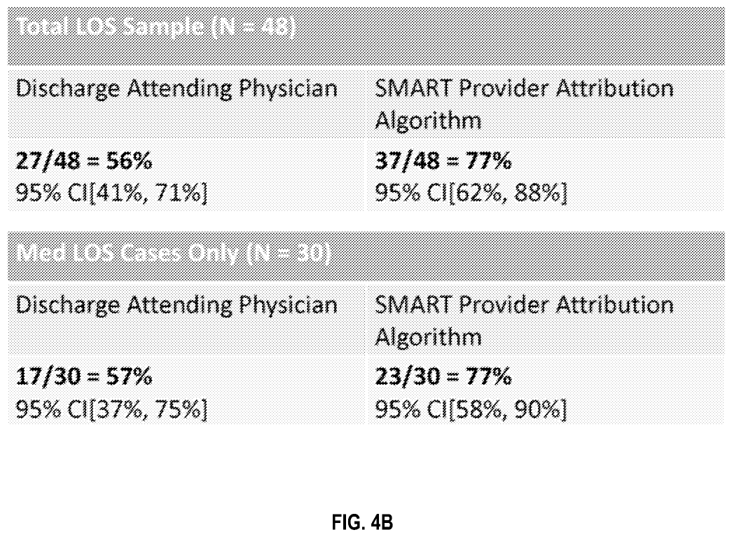

As shown by the above Example Score, the algorithm 103 can be leveraged to produce a score for each physician within each encounter associated with subsequent EHR data 166. Physicians can be ranked by respective scores (although the scores themselves are not comparable among encounters). For example, FIG. 3A illustrates the attribution output 150 can include a score 174A for a Clinician A, a score 174B for a Clinician B, and a score 174C for a Clinician C, all computed based on the subsequent EHR data 166 where each of the Clinicians A-C were involved in a new patient encounter. The attribution output 150 may further define a maximum score 176, which may apply to either of the Clinicians A-C based on their engagement with a patient as defined by the subsequent EHR data 166 when applied to the algorithm 103. In other words, once the algorithm 103 is formed and trained as described, the algorithm 103 can be leveraged to compute the attribution output 150 from the subsequent EHR data 166 by preprocessing the subsequent EHR data 166 to extract values associated with the predictor parameters 164, and then applying the extracted values to the algorithm 103. In some embodiments, the resultant attribution output 150 defines a plurality of scores associated with one or more physicians (Clinicians A-C in FIG. 3B) which improves attribution data analysis as opposed to assigning attribution based on an attending associated with the new encounter. FIGS. 4A-4B illustrate the efficacy of the algorithm 103 as an improvement over conventional attribution data analysis.

In addition, as reflected in block 210, attributing the physician most responsible for a patient's encounter and care may be used for a variety of metrics across different clinical systems, and for different means of reporting, including graphical reporting via the interface 154.

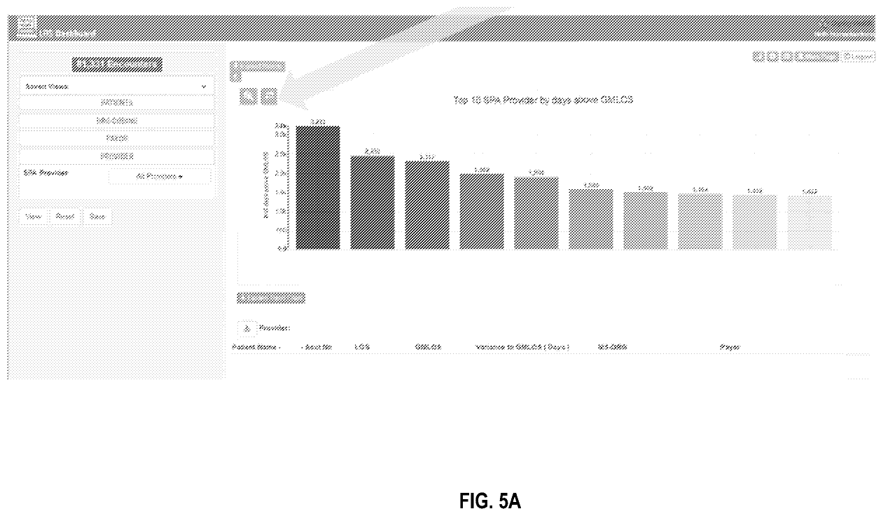

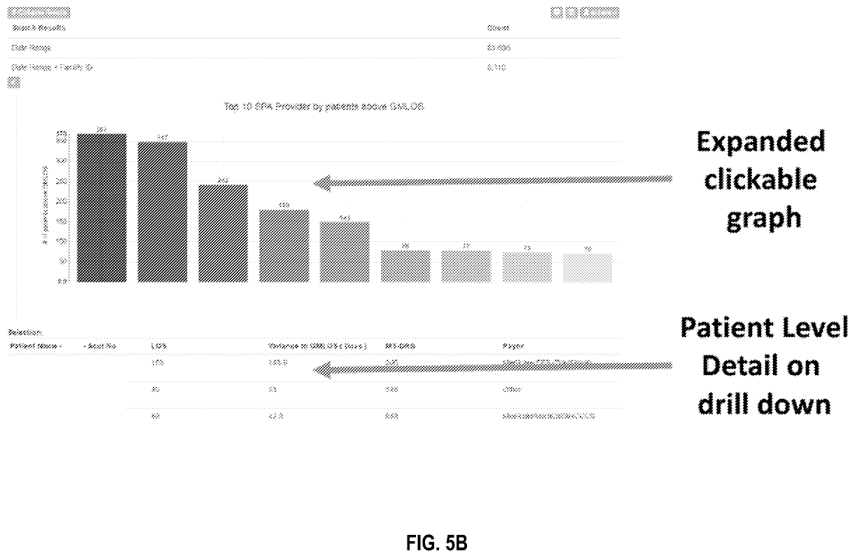

FIG. 5A and FIG. 5B are screenshots of the interface 154 of FIG. 3A. In general, FIG. 5A illustrates that the interface 154 can be leveraged to provide various forms of graphical reporting to any number of users to illustrate how physicians are being attributed from patient encounters. FIG. 5B illustrates that the interface 154 can be configured with drop-down detail options of graphs representative of the attribution output 150; i.e., the attribution output 150 can take many different forms as desired. The examples of the interface 154 are merely exemplary, non-limiting, and other aspects of the interface 154 are contemplated. Visualizing the attribution output 150 via the interface 154 as demonstrated further demonstrates the efficacy and value of the increased accuracy of attribution data analysis provided by formation and implementation of the algorithm 103.

Numerous related additional features are contemplated for formation and implementation of the algorithm 103. For example, the EHR data 112 may include any number or type of clinical records, financial records, or quality information and may be derived from clinical notes, surveys, and the like. In addition, the algorithm 103 is not limited to one particular machine learning or statistical model, and may include or be based on a plurality of machine learning or statistical models. In some embodiments, model averaging with resampling may be used to select a suitable/most parsimonious model. The algorithm 103 may be implemented by any number or combination of software languages or structures.

The functionality of the algorithm 103 may be implemented by way of a software application (e.g., application 511), provided as SaaS, a mobile app, a browser-based platform, and the like, and may include a graphical user interface (GUI) for displaying metrics and charting tools and other graphical aspects. Such an application may integrate basic information from clinical, financial, and quality data systems with an updated provider attribution algorithm to support analysis of Length of Stay for discharged patients, and is configured to provide an intuitive and efficient method to identify and analyze trends at the provider, service line, and facility level.

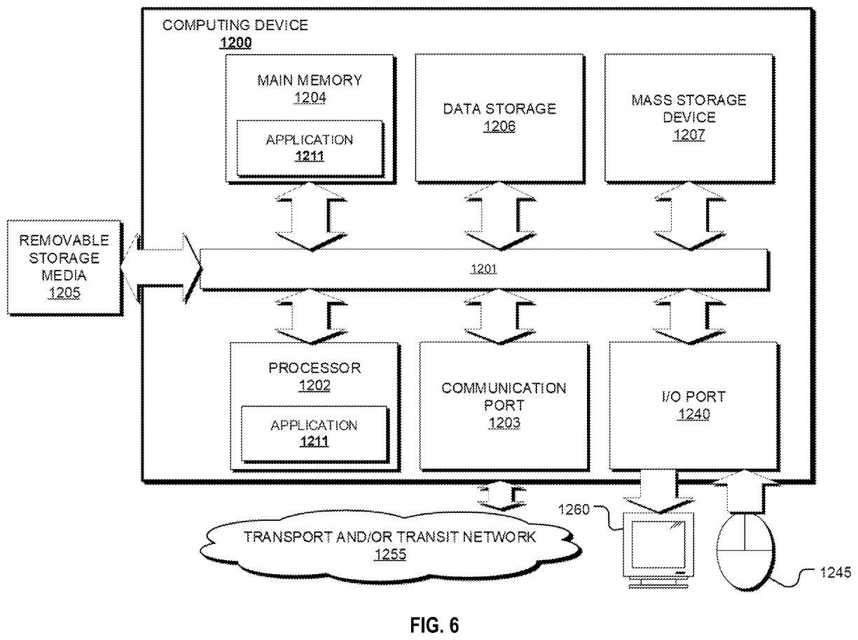

Referring to FIG. 6, a computing device 1200 is illustrated which may take the place of the computing device 102 and be configured, via an application 1211 and/or other software components, to execute functionality associated with the algorithm 103 and related aspects described herein, which may be translated to software or machine-level code, and may be installed to and/or executed by the computing device 1200 such that the computing device 1200 is configured to compute physician ranking attribution. It is contemplated that the computing device 1200 may include any number of devices, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments, and the like.

The computing device 1200 may include various hardware components, such as a processor 1202, a main memory 1204 (e.g., a system memory), and a system bus 1201 that couples various components of the computing device 1200 to the processor 1202. The system bus 1201 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computing device 1200 may further include a variety of memory devices and computer-readable media 1207 that includes removable/non-removable media and volatile/nonvolatile media and/or tangible media, but excludes transitory propagated signals. Computer-readable media 1207 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computing device 1200. Communication media includes computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The main memory 1204 includes computer storage media in the form of volatile/nonvolatile memory such as read only memory (ROM) and random access memory (RAM). A basic input/output system (BIOS), containing the basic routines that help to transfer information between elements within the computing device 1200 (e.g., during start-up) is typically stored in ROM. RAM typically contains data and/or program modules that are immediately accessible to and/or presently being operated on by processor 1202. Further, data storage 1206 in the form of Read-Only Memory (ROM) or otherwise may store an operating system, application programs, and other program modules and program data.

The data storage 1206 may also include other removable/non-removable, volatile/nonvolatile computer storage media. For example, the data storage 1206 may be: a hard disk drive that reads from or writes to non-removable, nonvolatile magnetic media; a magnetic disk drive that reads from or writes to a removable, nonvolatile magnetic disk; a solid state drive; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media provide storage of computer-readable instructions, data structures, program modules, and other data for the computing device 1200.

A user may enter commands and information through a user interface 1240 (displayed via a monitor 1260) by engaging input devices 1245 such as a tablet, electronic digitizer, a microphone, keyboard, and/or pointing device, commonly referred to as mouse, trackball or touch pad. Other input devices 1245 may include a joystick, game pad, satellite dish, scanner, or the like. Additionally, voice inputs, gesture inputs (e.g., via hands or fingers), or other natural user input methods may also be used with the appropriate input devices, such as a microphone, camera, tablet, touch pad, glove, or other sensor. These and other input devices 1245 are in operative connection to the processor 1202 and may be coupled to the system bus 1201, but may be connected by other interface and bus structures, such as a parallel port, game port or a universal serial bus (USB). The monitor 1260 or other type of display device may also be connected to the system bus 1201. The monitor 1260 may also be integrated with a touch-screen panel or the like.

The computing device 1200 may be implemented in a networked or cloud-computing environment using logical connections of a network interface 1203 to one or more remote devices, such as a remote computer. The remote computer may be a personal computer, a server, a router, a network PC, a peer device or other common network node, and typically includes many or all of the elements described above relative to the computing device 1200. The logical connection may include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computing device 1200 may be connected to a public and/or private network through the network interface 1203. In such embodiments, a modem or other means for establishing communications over the network is connected to the system bus 1201 via the network interface 1203 or other appropriate mechanism. A wireless networking component including an interface and antenna may be coupled through a suitable device such as an access point or peer computer to a network. In a networked environment, program modules depicted relative to the computing device 1200, or portions thereof, may be stored in the remote memory storage device.

Certain embodiments are described herein as including one or more modules. Such modules are hardware-implemented, and thus include at least one tangible unit capable of performing certain operations and may be configured or arranged in a certain manner. For example, a hardware-implemented module may comprise dedicated circuitry that is permanently configured (e.g., as a special-purpose processor, such as a field-programmable gate array (FPGA) or an application-specific integrated circuit (ASIC)) to perform certain operations. A hardware-implemented module may also comprise programmable circuitry (e.g., as encompassed within a general-purpose processor or other programmable processor) that is temporarily configured by software or firmware to perform certain operations. In some example embodiments, one or more computer systems (e.g., a standalone system, a client and/or server computer system, or a peer-to-peer computer system) or one or more processors may be configured by software (e.g., an application or application portion) as a hardware-implemented module that operates to perform certain operations as described herein.

Accordingly, the term “hardware-implemented module” encompasses a tangible entity, be that an entity that is physically constructed, permanently configured (e.g., hardwired), or temporarily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure the processor 1202, for example, to constitute a particular hardware-implemented module at one instance of time and to constitute a different hardware-implemented module at a different instance of time.

Hardware-implemented modules may provide information to, and/or receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and may store the output of that operation in a memory device to which it is communicatively coupled. A further hardware-implemented module may then, at a later time, access the memory device to retrieve and process the stored output. Hardware-implemented modules may also initiate communications with input or output devices.

Computing systems or devices referenced herein may include desktop computers, laptops, tablets e-readers, personal digital assistants, smartphones, gaming devices, servers, and the like. The computing devices may access computer-readable media that include computer-readable storage media and data transmission media. In some embodiments, the computer-readable storage media are tangible storage devices that do not include a transitory propagating signal. Examples include memory such as primary memory, cache memory, and secondary memory (e.g., DVD) and other storage devices. The computer-readable storage media may have instructions recorded on them or may be encoded with computer-executable instructions or logic that implements aspects of the functionality described herein. The data transmission media may be used for transmitting data via transitory, propagating signals or carrier waves (e.g., electromagnetism) via a wired or wireless connection.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method of computing physician attribution, comprising:
accessing, by a processor, a training dataset including electronic health record (EHR) data defining predefined decisions for physician attribution; and
training, by machine learning conducted by the processor, a machine learning algorithm to learn and model the predefined decisions for physician attribution in view of the training dataset such that the processor executing the machine learning algorithm is configured to predict physician attribution from subsequent EHR data, by:
inputting a plurality of variables associated with the predefined decisions of the physician attribution to a machine learning model,
learning a set of predictor parameters for the machine learning algorithm that improve physician attribution prediction, and
weighting and summing one or more fields of the machine learning algorithm in view of an accuracy threshold,
wherein the machine learning algorithm as trained, when fed with the subsequent EHR data defining a patient encounter, improves attribution data analysis by computing an attribution output based on the set of predictor parameters as learned.

2. The method of claim 1, wherein the plurality of variables includes a physician type, a physician status, and a length of stay associated with a record of a past encounter with a physician predetermined to be properly attributed.

3. The method of claim 1, wherein the set of predictor parameters includes daily progress notes per day, long notes per day, orders per day, a length of stay, and an attending parameter.

4. The method of claim 1, further comprising, by the processor, performing LASSO regression to select the set of predictor parameters via leave-one-encounter-out cross validation.

5. The method of claim 1, further comprising, by the processor, evaluating accuracy of the machine learning algorithm by feeding the machine learning algorithm with information from a validation set defining additional predefined decisions for physician attribution and associated variables.

6. The method of claim 1, wherein the machine learning algorithm includes a regression algorithm that models relationships between the plurality of variables to select the set of predictor parameters, and the machine learning algorithm is trained by the processor iteratively by refining the machine learning algorithm using a measure of error associated with a score of the attribution output.

7. The method of claim 1, wherein the machine learning algorithm considers all two-way interactions between a patient and a physician.

8. A system for computing physician attribution, comprising:
a first computing device, the first computing device having access to electronic healthcare records (EHR) data; and
a second computing device in operable communication with the first computing device, the second computing device including a processor configured to:
access the EHR data from the first computing device,
generate a training dataset from the EHR data including predefined decisions of physician attribution,
input a plurality of variables associated with the predefined decisions of the physician attribution to a machine learning model, and
generate and train a machine learning algorithm based on the machine learning model such that the machine learning algorithm is configured to learn the predefined decisions of physician attribution and is configured to predict physician attribution from subsequent EHR data.

9. The system of claim 8, wherein the machine learning model is a regression model, and the second computing device applies the plurality of variables to the machine learning model to generate a set of predictor-parameters for the machine learning algorithm.

10. The system of claim 9, wherein the regression model includes at least one of linear regression, logistic regression, polynomial regression, stepwise regression, ridge regression, LASSO regression, or ElasticNet regression.

11. The system of claim 9, wherein the processor of the second computing device applies a weight to at least one the set of predictor-parameters to optimize a predetermined accuracy threshold for the machine learning algorithm.

12. A tangible, non-transitory, computer-readable media having instructions encoded thereon, the instructions, when executed by a processor, are operable to:
access EHR data,
generate a training dataset from the EHR data including predefined decisions of physician attribution,
input a plurality of variables associated with the predefined decisions of the physician attribution to a machine learning model, and
train a machine learning algorithm based on the machine learning model such that the machine learning algorithm is configured to learn the predefined decisions of physician attribution and is configured to predict physician attribution from subsequent EHR data.

13. The tangible, non-transitory, computer-readable media of claim 12, wherein the machine learning model executed by the processor finds casual effect relationships between variables of the plurality of variables.

14. The tangible, non-transitory, computer-readable media of claim 12, further comprising additional instructions that when executed by the processor are operable to:
select, by the input of the plurality of variables to the machine learning model, a set of predictor-parameters from the plurality of variables.

15. The tangible, non-transitory, computer-readable media of claim 14, further comprising additional instructions that when executed by the processor are operable to:
apply a regularization method and shrink one more coefficients of the machine learning model to zero to improve feature selection of a set of parameter-predictors for the machine learning algorithm.
